(19) United States
(12) Patent Application Publication
(10) Pub. No.: US 2026/0108379 A1
(43) Pub. Date: Apr. 23, 2026

(54) TONGUE TRAINING DEVICE

(71) Applicant: KAOHSIUNG MEDICAL UNIVERSITY, Kaohsiung (TW)

(72) Inventors: Chia-Hsin Chen, Kaohsiung (TW); Yi-Hung Pai, Kaohsiung (TW)

(21) Appl. No.: 18/920,950

(22) Filed: Oct. 20, 2024

(51) Int. Cl. A61F 5/56; A61M 16/00

(52) U.S. Cl. CPC A61F 5/566; A61M 16/0009; A61M 2205/3331; A61M 2205/3592; A61M 2205/52; A61M 2210/0643

(57) ABSTRACT

The present invention provides a tongue training device, which includes an elastic body, a displacement sensor and a wireless communicator. Wherein, the elastic body has a tongue inlet and an accommodating space in communication with the tongue inlet, so as to accommodate a part or a whole of the tongue of the user in the accommodating space. The displacement sensor is disposed on the elastic body to sense the displacement distance and the displacement direction of the elastic body, and generate the corresponding displacement distance signal and displacement direction signal. And, the wireless communicator is disposed on the elastic body, connects the displacement sensor and the external device, and transmits the displacement distance signal and the displacement direction signal to the external device, so as to operate the external device according to the displacement distance signal and the displacement direction signal.

### BACKGROUND OF THE INVENTION

### 1. Field of the Invention

The present disclosure generally relates to a training device, in particular to a tongue training device, which is configured to sense and record the movement of the tongue of the user, and operate an external device based on the movement of the tongue of the user.

### 2. Description of the Related Art

Obstructive sleep apnea (OSA) is a common sleep disorder in modern society, which may cause serious harm to people's health. Obstructive sleep apnea may damage the white matter in the brain, and may lead to an increased risk of cardiovascular diseases, such as hypertension, heart disease and stroke, as well as diabetes, and may even cause depression. In the United States, about 30 million people have symptoms of obstructive sleep apnea, which seriously affects people's productivity and increases the risk of accidents due to inattention. It is estimated to cost more than $150 billion annually in social and medical costs in the United States.

Wherein, tongue hypertrophy may lead to nocturnal snoring, choking, or intermittent apnea, and people with sleep apnea symptoms may also have a hypertrophied tongue and a higher fat percentage of the tongue. Therefore, tongue muscle training to reducing the fat rate of the tongue may effectively improve the symptoms of obstructive sleep apnea.

The conventional training device for tongue muscle usually fixes the user's tongue by adsorption, and stretches the tongue to achieve the effect of training the tongue muscles. However, in this training method, the external force of stretching the tongue can only be manually controlled by a physical therapist. In the case of the force applied is too large, it may cause discomfort to the user, and in the case of the force applied is too small, it may be difficult to achieve the effect of training. In addition, in the training method using the conventional tongue training device, since the training intensity is controlled manually, the entire training process cannot be data-recorded, and the training intensity can only be adjusted by relying on the experience of the physical therapist.

In addition, the conventional tongue-controlled input devices, such as a mouse, requires the user to press a specific function key on the input device with the tongue to complete the operation of the external device. Further, the control process does not require significant movement of the tongue, and thus it is of limited help in tongue training.

In summary, the inventor of the present disclosure conceived and designed a tongue training device, which may provide tongue movement training, store tongue movement records, and operate external devices simultaneously, thereby enhancing the implementation and utilization in the industry.

### SUMMARY OF THE INVENTION

In view of the above problems, the present disclosure is to provide a tongue training device.

To achieve the foregoing objective, the present disclosure provides a tongue training device, including an elastic body, a displacement sensor, and a wireless communicator. Wherein, the elastic body includes a tongue inlet and an accommodating space in communication with the tongue inlet, so as to accommodate a part or a whole of a tongue of a user in the accommodating space. The displacement sensor is disposed on the elastic body to sense a displacement distance and a displacement direction of the elastic body, and generate a corresponding displacement distance signal and a displacement direction signal. And, the wireless communicator is disposed on the elastic body, connected between the displacement sensor and an external device, and transmits the displacement distance signal and the displacement direction signal to the external device, so as to perform an operation on the external device based on the displacement distance signal and the displacement direction signal. Wherein, a length is defined between an end part of the elastic body located at the tongue inlet and an opposite end part away from the end part, the aspect ratio of the width of the end part to the length is at least greater than 1.5, and the elastic body has at least one section of a fixed width between the end part and the opposite end part. And, a negative pressure space is generated inside the accommodating space by pressing the elastic body, so that the tongue be adsorbed by the elastic body due to elastic recovery of the elastic body.

In a preferred embodiment of the present disclosure, the elastic body may include a v-shaped recess, which is disposed around the tongue inlet corresponding to the position of a frenulum linguae of the tongue.

In a preferred embodiment of the present disclosure, the elastic body may have a connection structure, which is provided at the opposite end part of the elastic body away from the tongue inlet. One end of the connection structure is fixed to the elastic body, and the other end detachably combines the displacement sensor and the wireless communicator.

In a preferred embodiment of the present disclosure, the other end of the connection structure may be provided with at least one of groove to detachably fix the displacement sensor and the wireless communicator.

In a preferred embodiment of the present disclosure, the operation may include performing at least one of the following actions: controlling the movement of a cursor displayed by the external device; executing an object selected by the cursor; and generating and storing a tongue movement record based on a received signal.

In a preferred embodiment of the present disclosure, the external device may generate the tongue movement record based on the displacement distance signal and the displacement direction signal, and the tongue movement record includes the following parameters: displacement direction, displacement distance, displacement duration, displacement speed, and displacement frequency.

In a preferred embodiment of the present disclosure, the tongue training device may further include a pressure sensor, which is provided at one end of the elastic body, and the pressure sensor senses the occlusal action of human body to generate a corresponding occlusal signal. Wherein, the wireless communicator may connect the pressure sensor and the external device, and transmits the occlusal signal to the external device to perform the operation on the external device based on the occlusal signal.

In a preferred embodiment of the present disclosure, the external device may generate the tongue movement record based on the occlusal signal, and the tongue movement record includes the following parameters: occlusal pressure and occlusal frequency.

In a preferred embodiment of the present disclosure, the displacement sensor may include a three-axis gyroscope or a six-axis gyroscope.

In a preferred embodiment of the present disclosure, the wireless communication method of the wireless communicator may be selected from at least one of the group consisting of narrow-band Internet of Things, 3G, 4G, 5G, Long-Term Evolution, wireless radio frequency communication, infrared communication, Bluetooth, Zigbee and Wi-Fi.

In summary, the tongue training device of the present disclosure is provided with the displacement sensor to sense the tongue movement status of the user, so as to generate the corresponding displacement distance signal and displacement direction signal. And, the tongue training device of the present disclosure is provided with the pressure sensor to sense the occlusal action of the user, so as to generate the corresponding occlusal signal. Further, the above-mentioned signals corresponding to the tongue movement status and the occlusal action may be transmitted to an external device through the wireless communicator, and thus, the external device may be used to store the signals related to the tongue movement status and the occlusal action, and the external device may be operated based on the above-mentioned signals related to the tongue movement status and occlusal action. For example, the displacement distance signal and displacement direction signal may be used to control the movement of the cursor displayed by the external device, and the occlusal signal may be used to control the cursor displayed by the external device to click on the selected object. Through this configuration, the tongue training device of the present disclosure may achieve the technical effects of performing tongue muscle movement training, storing tongue movement records, and operating external devices.

### BRIEF DESCRIPTION OF THE DRAWINGS

The features and technical contents of the present disclosure will be further appreciated and understood with reference to the detailed description of preferred embodiments and accompanying drawings.

FIG. 1 is a schematic diagram of the elastic body of the tongue training device in accordance with an embodiment of the present disclosure.

FIG. 2 is a schematic diagram of the tongue training device in accordance with an embodiment of the present disclosure.

FIG. 3 is a schematic block diagram of the tongue training device in accordance with an embodiment of the present disclosure.

FIG. 4 is a schematic diagram of the tongue training device in accordance with another embodiment of the present disclosure.

### DETAILED DESCRIPTION OF THE PREFERRED EMBODIMENTS

In order to facilitate the understanding of the technical features, contents and advantages of the present disclosure and the effective thereof that can be achieved, the present disclosure will be illustrated in detail below in the form of embodiments with the accompanying drawings. The diagrams used herein are merely intended to be schematic and auxiliary to the specification, but are not necessary to be true scale and precise to the configuration after implementing the present disclosure. Thus, it should not be interpreted in accordance with the scale and the configuration of the accompanying drawings to limit the scope of the present disclosure on the practical implementation.

The advantages, features, and technical methods of accomplishing the present disclosure will be more easily understood by being described in more detail with reference to the exemplary embodiments and the accompanying drawings, and the present disclosure may be implemented in different forms, so it should not be construed as being limited to the content described herein. Rather, these embodiments are provided as examples so that this disclosure will be thorough and complete, and will fully convey the aspects of the present disclosure to those skilled in the art, and the present disclosure will only be defined by the appended claims.

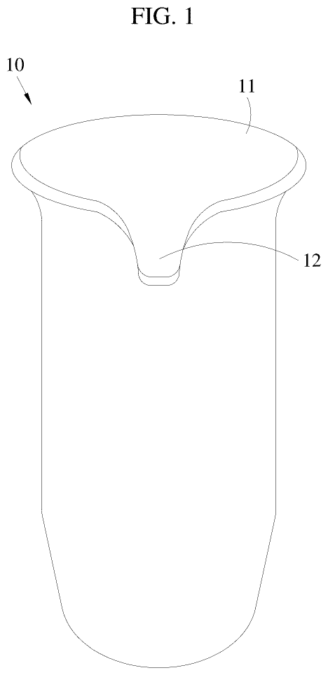

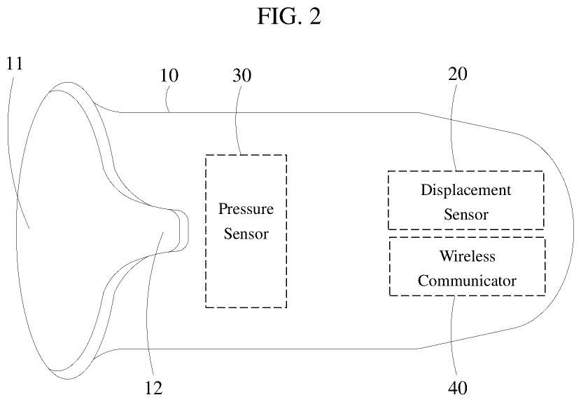

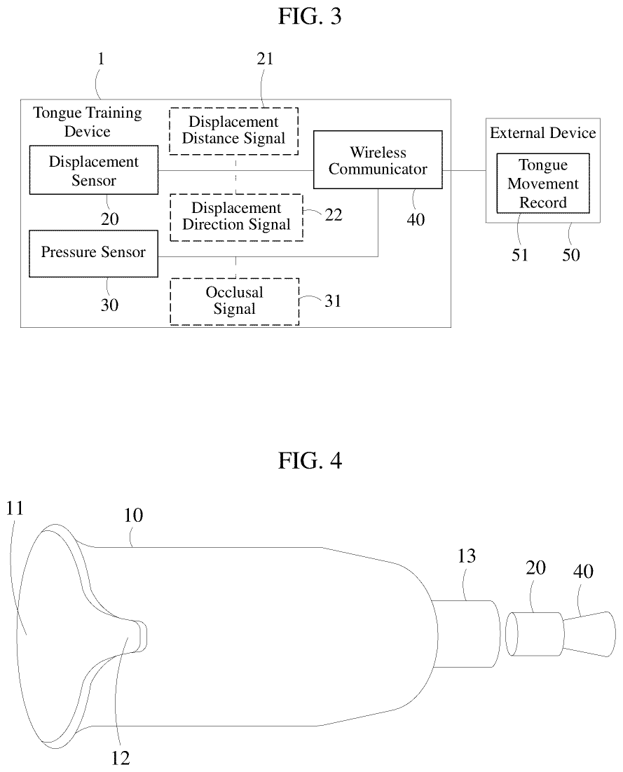

Referring to FIG. 1 to FIG. 3, FIG. 1 is a schematic diagram of the elastic body of the tongue training device in accordance with an embodiment of the present disclosure, FIG. 2 is a schematic diagram of the tongue training device in accordance with an embodiment of the present disclosure, and FIG. 3 is a schematic block diagram of the tongue training device in accordance with an embodiment of the present disclosure.

As shown in FIG. 1 to FIG. 3, the present disclosure provides a tongue training device 1, including an elastic body 10, a displacement sensor 20, and a wireless communicator 40. Wherein, the elastic body 10 includes a tongue inlet 11 and an accommodating space in communication with the tongue inlet 11, so as to accommodate a part or the whole of a tongue of the user in the accommodating space. The displacement sensor 20 is disposed on the elastic body 10 to sense a displacement distance and a displacement direction of the elastic body 10, and generate a corresponding displacement distance signal 21 and a displacement direction signal 22. And, the wireless communicator 40 is disposed on the elastic body 10, connected between the displacement sensor 20 and an external device 50, and configured to transmit the displacement distance signal 21 and the displacement direction signal 22 to the external device 50, so as to perform a predetermined operation on the external device 50 based on the displacement distance signal 21 and the displacement direction signal 22.

Wherein, the elastic body 10 includes a substantially v-shaped recess 12, which is disposed around the tongue inlet 11 corresponding to the position of a frenulum linguae of the tongue. With the configuration of the v-shaped recess 12 of the elastic body 10, the user's tongue may be easily accommodated in the accommodating space of the elastic body 10 through the tongue inlet 11, and will not be blocked by the frenulum linguae of the tongue against the tongue inlet 11 of the elastic body 10. Wherein, as shown in FIG. 1, the elastic body 10 has at least one section of a fixed width between an end part of the elastic body 10 located at the tongue inlet 11 and the opposite end part away from the end part. Specifically, a negative pressure space may be generated inside the accommodating space by pressing the elastic body 10, so that the user's tongue may be adsorbed by the elastic body 10 due to the elastic recovery of the elastic body 10. Therefore, this configuration may reduce the occurrence of the tongue training device 1 of the present disclosure falling off from the user's tongue during the training process. In the present embodiment, the aspect ratio of the elastic body 10 is approximately 2, and the present disclosure is not limited to the above embodiments. In other embodiments, the aspect ratio of the elastic body 10 is at least greater than 1.5, so that the elastic body may provide better adsorption force.

Furthermore, in the present embodiment, the displacement sensor 20 provided with a gyroscope as an acceleration sensor, which is disposed on the elastic body 10 and at the end away from the tongue inlet 11 to sense the movement status of the user's tongue. In the present embodiment, a three-axis gyroscope is used as the acceleration sensor of the displacement sensor 20, and the present disclosure is not limited to the above embodiments. In other embodiments, other suitable sensing elements such as a six-axis gyroscope may be provided based on the requirement to sense the movement status of the user's tongue.

Specifically, the displacement sensor 20 may use a three-axis gyroscope to sense the movement of the user's tongue in at least six directions of up, down, left, right, front and back, to generate a corresponding displacement direction signal 22. Further, the displacement sensor 20 may sense the acceleration value of each movement of the user's tongue to generate the displacement distance signal 21 containing the acceleration value, so that the external device 50 may calculate the displacement distance of each movement based on the acceleration value in the received displacement distance signal 21. Furthermore, the wireless communicator 40 may transmit the above-mentioned displacement distance signal 21 and displacement direction signal 22 to the external devices 50, such as desktop computers, tablet computers, and smart phones, through wireless communication, so that the tongue training device 1 of the present disclosure may be used as a controller, such as a mouse, to operate the external device 50 with the displacement distance signal 21 and the displacement direction signal 22 generated through each movement of the tongue, thereby controlling the movement of the cursor displayed by the external device 50.

In addition, in the present embodiment, the tongue training device 1 may further include a pressure sensor 30, which is disposed at the opposite end part of the elastic body 10 and at a position convenient for the user's teeth to bite. The pressure sensor 30 includes at least one pressure sensor to sense the occlusal action of the user's teeth and generate a corresponding occlusal signal 31. Furthermore, the wireless communicator 40 transmits the above-mentioned occlusal signal 31 to the external devices 50, such as desktop computers, tablet computers, and smart phones, through wireless communication, so that the tongue training device 1 of the present disclosure may be used as a controller, such as a mouse, to operate the external device 50 with the occlusal signal 31 generated through each occlusal action of the user, thereby controlling the movement of the cursor displayed by the external device 50.

It should be noted that, since the above-mentioned displacement distance signal 21, displacement direction signal 22 and occlusal signal 31 are all transmitted to the external device 50 through the wireless communicator 40, the external device 50 may generate the corresponding tongue movement record 51 based on the received above-mentioned displacement distance signal 21, displacement direction signal 22 and occlusal signal 31. Specifically, the tongue movement record 51 may include at least one of the following parameters: displacement direction, displacement distance, displacement duration, displacement speed, displacement frequency, occlusal pressure, and occlusal frequency. Through this configuration, the tongue training device 1 of the present disclosure may generate a tongue movement record 51 to record the movement pattern and amount of motion the user's tongue during the entire tongue training process. In addition to being a reference for physical therapists to adjust the training details of tongue training accordingly, the tongue movement record 51 may also allow users to see the effectiveness of training through visible data, so as to enhance user's confidence and motivation to perform training.

That is, With the configuration of the displacement sensor 20, pressure sensor 30, and wireless communicator 40, the tongue training device 1 of the present disclosure may perform predetermined operations on the external device 50 based on the movement status of the user's tongue. Wherein, the above-mentioned operations performed on the external device 50 include performing at least one of the following actions: controlling the movement of the cursor displayed by the external device 50; executing an object selected by the cursor; and generating and storing the tongue movement record 51 based on the received signal (displacement distance signal, displacement direction signal, and occlusal signal).

In addition, in the present embodiment, the wireless communication method of the wireless communicator 40 uses Bluetooth communication technology, and the present disclosure is not limited to the above embodiments. In other embodiments, the wireless communication method of the wireless communicator may be selected from at least one of the group consisting of narrow-band Internet of Things, 3G, 4G, 5G, Long-Term Evolution, wireless radio frequency communication, infrared communication, Bluetooth, Zigbee and Wi-Fi.

Referring to FIG. 4, FIG. 4 is a schematic diagram of the tongue training device in accordance with another embodiment of the present disclosure.

In the present embodiment, the tongue training device 1 has a similar configuration to the tongue training device in the above embodiment, the tongue training device 1 of the present embodiment also provided with a displacement sensor 20 and a wireless communicator 40, but is not provided with a pressure sensor. Further, the tongue training device 1 of the present embodiment is also connected to the external device 50 through wireless communication to achieve the technical effects of performing tongue movement training, storing the tongue movement record 51, and operating the external device 50. The other specific configuration of the tongue training device 1 of the present embodiment is as disclosed in the above embodiment, so the present embodiment will not go into details here.

As shown in FIG. 4, in the present embodiment, the elastic body 10 of the tongue training device 1 also includes a v-shaped recess 12 to accommodate the frenulum linguae of the tongue. It should be noted that, in the present embodiment, the elastic body 10 may further include a connection structure 13, which is disposed at the opposite end part of the elastic body 10 away from the tongue inlet 11, and one end of the connection structure 13 is fixed to the elastic body 10, and the other end of the connection structure 13 may be detachably combined with the displacement sensing. Specifically, one end of the connection structure 13 of the elastic body 10 may be fixed to the elastic body 10, and the other end may be provided with a circular groove, and the inner diameter of the circular groove may be correspond to the outer diameter of the displacement sensor 20 with cylindrical shape, so that the displacement sensor 20 may be detachably inserted and fixed into the connection structure 13 of elastic body 10. In addition, in the present embodiment, the displacement sensor 20 and the wireless communicator 40 may be formed into an integrated structure, and thus the displacement sensor 20 may be fixed to the connection structure 13 together with the wireless communicator 40. Further, the wireless communicator 40 may be exposed to the outside of the elastic body 10, so as to be beneficial to the transmission of signals, and the present disclosure is not limited to the above embodiments. In other embodiments, the connection structure of the elastic body may have other types of configurations to detachably combine the displacement sensor and the wireless communicator. For example, the connection structure may have two grooves to accommodate the displacement sensor and the wireless communicator respectively.

In summary, the tongue training device of the present disclosure is provided with the displacement sensor to sense the tongue movement status of the user, so as to generate the corresponding displacement distance signal and displacement direction signal. And, the tongue training device of the present disclosure may be provided with the pressure sensor to sense the occlusal action of the user, so as to generate the corresponding occlusal signal. Further, the above-mentioned signals corresponding to the tongue movement status and the occlusal action may be transmitted to an external device through the wireless communicator, and thus, the external device may be used to store the signals related to the tongue movement status and the occlusal action, and the external device may be operated based on the above-mentioned signals related to the tongue movement status and occlusal action. For example, the displacement distance signal and displacement direction signal may be used to control the movement of the cursor displayed by the external device, and the occlusal signal may be used to control the cursor displayed by the external device to click on the selected object. Through this configuration, the tongue training device of the present disclosure may achieve the technical effects of performing tongue muscle movement training, storing tongue movement records, and operating external devices.

The above-mentioned embodiments are only to illustrate the technical concept and features of the present disclosure, and for those having ordinary skill in the art to understand the content of the present disclosure and implement the present disclosure accordingly. It should not be construed as limiting the scope of the present disclosure. It is to be construed that all changes or modifications derived from the spirit of the present disclosure are included in the scope of the disclosure.

What is claimed is:

1. A tongue training device, including:
an elastic body, including a tongue inlet and an accommodating space in communication with the tongue inlet, so as to accommodate a part or a whole of a tongue of a user in the accommodating space;
a displacement sensor, disposed on the elastic body and configured to sense a displacement distance and a displacement direction of the elastic body, and to generate a corresponding displacement distance signal and a displacement direction signal; and
a wireless communicator, disposed on the elastic body, connected between the displacement sensor and an external device, and configured to transmit the displacement distance signal and the displacement direction signal to the external device, so as to perform an operation on the external device based on the displacement distance signal and the displacement direction signal;
wherein a length is defined between an end part of the elastic body located at the tongue inlet and an opposite end part away from the end part, an aspect ratio of an width of the end part to the length is at least greater than 1.5, the elastic body has at least one section of a fixed width between the end part and the opposite end part, and a negative pressure space is generated inside the accommodating space by pressing the elastic body, so that the tongue is adsorbed by the elastic body due to elastic recovery of the elastic body.

2. The tongue training device of claim 1, wherein the elastic body including:
a v-shaped recess, which is disposed around the tongue inlet corresponding to the position of a frenulum linguae of the tongue.

3. The tongue training device of claim 1, wherein the elastic body has a connection structure, which is provided the opposite end part of the elastic body away from the tongue inlet, one end of the connection structure is fixed to the elastic body, and one other end detachably combines the displacement sensor and the wireless communicator.

4. The tongue training device of claim 3, wherein the other end of the connection structure is provided with at least one of groove to detachably fix the displacement sensor and the wireless communicator.

5. The tongue training device of claim 1, wherein the operation includes performing at least one of the following actions: controlling the movement of a cursor displayed by the external device; executing an object selected by the cursor; and generating and storing a tongue movement record based on a received signal.

6. The tongue training device of claim 5, wherein the external device generates the tongue movement record based on the displacement distance signal and the displacement direction signal, and the tongue movement record includes the following parameters: displacement direction, displacement distance, displacement duration, displacement speed, and displacement frequency.

7. The tongue training device of claim 5, wherein the tongue training device further includes a pressure sensor, which is provided at one end of the elastic body, and the pressure sensor senses an occlusal action of human body to generate a corresponding occlusal signal;
wherein, the wireless communicator connects the pressure sensor and the external device, and transmits the occlusal signal to the external device to perform the operation on the external device based on the occlusal signal.

8. The tongue training device of claim 7, wherein the external device generates the tongue movement record based on the occlusal signal, and the tongue movement record includes the following parameters: occlusal pressure and occlusal frequency.

9. The tongue training device of claim 1, wherein the displacement sensor includes a three-axis gyroscope or a six-axis gyroscope.

10. The tongue training device of claim 1, wherein the wireless communication method of the wireless communicator is selected from at least one of the group consisting of narrow-band Internet of Things, 3G, 4G, 5G, Long-Term Evolution, wireless radio frequency communication, infrared communication, Bluetooth, Zigbee and Wi-Fi.
